(19) United States
(12) Patent Application Publication
(10) Pub. No.: US 2024/0002798 A1
(43) Pub. Date: Jan. 4, 2024

(54) METHOD FOR PREPARING IMMUNOGENICITY-ENHANCED CD103+ FCGR3+ DENDRITIC CELL BY TREATMENT WITH INTERLEUKIN-33 AND PHARMACEUTICAL COMPOSITION COMPRISING SAME DENDRITIC CELL FOR CANCER IMMUNOTHERAPY

(71) Applicant: Research & Business Foundation Sungkyunkwan University, Suwon-si (KR)

(72) Inventors: Yong-Soo BAE, Suwon-si (KR); Myeongho KANG, Daegu (KR); Jung-Hyub HONG, Chilgok-gun (KR)

(73) Assignee: Research & Business Foundation Sungkyunkwan University, Suwon-si (KR)

(21) Appl. No.: 18/037,293

(22) PCT Filed: Nov. 16, 2021
(86) PCT No.: PCT/KR2021/016813

§ 371 Date: May 16, 2023

(30) Foreign Application Priority Data
Nov. 17, 2020 (KR) 10-2020-0153308
Nov. 16, 2021 (KR) 10-2021-0158012

(51) Int. Cl. C12N 5/0784; A61K 35/15; A61P 35/00

(52) U.S. Cl. CPC C12N 5/0639; A61K 35/15; A61P 35/00

(57) ABSTRACT

The present disclosure relates to a method for increasing anti-tumor immunity of a dendrocytic therapeutic agent and, more specifically, to a method for preparing cluster of differentiation 103 (CD103)-positive dendritic cells, CD103-positive dendritic cells prepared by the preparation method, and a pharmaceutical composition and kit comprising same for cancer immunotherapy, the method comprising a step of treating dendrocyte progenitor cells with interleukin-33 (IL-33) when the dendrocyte progenitor cells are cultured in a medium comprising FMS-like tyrosine kinase 3 ligand (Flt3L) and thus differentiated to dendritic cells. As the dendritic cells differentiated by the preparation method of the present disclosure exhibit a high potential of inducing antigen-specific cytotoxic T cells, compared to control dendritic cells, and antitumor immunity is strongly induced by a subpopulation of the newly differentiated dendritic cells, the preparation method of the present disclosure has the advantage of increasing immunogenicity in the differentiation step in contrast to a convention method of increasing immunogenicity after completion of differentiation to dendritic cells. Therefore, it is expected that novel immune cell therapy capable of greatly increasing a therapeutic effect through the preparation method of the present disclosure and dendritic cells cultured ex vivo using same can be provided.

### TECHNICAL FIELD

The present disclosure relates to a method for enhancing antitumor immunity of a dendritic cell therapeutic agent. Specifically, the present disclosure relates to a method for preparing cluster of differentiation 103 (CD103)-positive dendritic cells, CD103-positive dendritic cells prepared by the method, and a pharmaceutical composition and a kit each including the same for cancer immunotherapy, the method includes culturing dendritic progenitor cells in a medium comprising FMS-like tyrosine kinase 3 ligand (Flt3L) to induce the dendritic progenitor cells to differentiate into dendritic cells, wherein a treatment with interleukin-33 (IL-33) is performed in a stage of differentiation into the dendritic cells.

This application claims priorities to and the benefits of Korean Patent Application No. 10-2020-0153308 filed on 17 Nov. 2020 and Korean Patent Application No. 10-2021-0158012 filed on 16 Nov. 2021, the disclosure of which is incorporated herein by reference.

### BACKGROUND ART

Dendritic cells, which are typical antigen-presenting cells, can activate cytotoxic T cells by presenting antigens to the cytotoxic T cells, and cell therapeutic agents for tumor therapy are being actively developed by the application of dendritic cells.

Due to an insufficient number of in vivo dendritic cells, in vitro cultured dendritic cells are used for tumor therapy. In most cases, granulocyte-macrophage colony-stimulating factor (GM-CSF) has been used for the preparation of existing in vitro cultured dendritic cells, and in recent years, FMS-like tyrosine kinase 3 ligand (Flt3L) is used to prepare cells more similar to in vivo dendritic cells. Genetic modification is attempted or adjuvants are used to enhance immunogenicity of these GM/FL-BMDC, but there are unexpected side effects, such as off-target effects or excessive inflammation caused by inflammatory cytokines. Moreover, the methods suggested above influence dendritic cells at the time of completion of differentiation, focusing only on an increase in the expression of specific co-stimulatory molecules and a change in cytokines capable of stimulating T cells.

As such, most in-vitro cultured dendritic cell therapeutic agents are aimed at enhancing immunogenicity after the completion of differentiation, and research for forming new dendritic cell subsets and enhancing immunogenicity by controlling an intermediate stage of differentiation is almost at the laboratory level. Moreover, even dendritic cell therapeutics showing significant effects in vitro experiments do not show tumor inhibitory effects since the cells are inactivated due to immune checkpoint molecules increased by in vivo tumor environments. Therefore, attempts are being made to overcome the above problem by developing antibodies against immune checkpoint molecules, but there are limited cases of tumors to be applicable. Research on the development of dendritic cell therapeutic agents for intractable cancer, of which safety was proven as autologous cell therapeutic agents, has been greatly advanced for last 20 years, but antitumor immunity inducing ability under expectations and limited therapy effects still remain to be addressed.

### DISCLOSURE OF INVENTION

### Technical Problem

To fundamentally enhance the immunogenicity of in vitro cultured dendritic cells, the present inventors revealed dendritic cell subsets that are newly induced by interleukin-33 (IL-33) and established that anti-tumor immunity induced by the dendritic cell subsets is significantly higher than that of conventional dendritic cell therapeutic agents, and thus attempted to develop a method for manufacturing more effective dendritic cell therapeutic agents.

Accordingly, an aspect of the present disclosure is to provide a method for preparing cluster of differentiation 103 (CD103)-positive dendritic cells, the method includes culturing dendritic progenitor cells in a medium comprising FMS-like tyrosine kinase 3 ligand (Flt3L) to induce the dendritic progenitor cells to differentiate into dendritic cells, wherein a treatment with interleukin-33 is performed in a stage of differentiation into the dendritic cells.

Another aspect of the present disclosure is to provide CD103-positive dendritic cells prepared by the method according to the present disclosure.

Still another aspect of the present disclosure is to provide a pharmaceutical composition for cancer immunotherapy, the composition comprising CD103-positive dendritic cells prepared by the method according to the present disclosure.

Still another aspect of the present disclosure is to provide a kit for cancer immunotherapy, the kit including as one element CD103-positive dendritic cells obtained by the preparation method according to the present disclosure.

Still another aspect of the present disclosure is to provide a method for assessing the immunogenicity of dendritic cells, the method includes measuring the proportion of CD103-positive dendritic cells.

However, the problems to be solved by the present disclosure are not limited to the above-mentioned problems, and other problems not mentioned would be clearly understood by a person skilled in the art from the following description.

### Solution to Problem

In accordance with an aspect of the present disclosure, there is provided a method for preparing cluster of differentiation 103 (CD103)-positive dendritic cells, the method includes culturing dendritic progenitor cells in a medium comprising FMS-like tyrosine kinase 3 ligand (Flt3L) to induce the dendritic progenitor cells to differentiate into dendritic cells, wherein a treatment with interleukin-33 (IL-33) is performed in a stage of differentiation into the dendritic cells.

The present inventors induced CD103-positive dendritic cells with excellent immunogenicity by performing a treatment with IL-33 in a stage of differentiation into dendritic cells, and verified that such induced dendritic cells had more effective tumor inhibitory efficacy than dendritic cell medicines obtained by existing differentiation methods and thus completed the present disclosure. Furthermore, the present inventors verified that a direct administration of IL-33 into experimental animals resulted in tumor inhibitory efficacy and developed the present disclosure by confirming the induction of new CD103-positive dendritic cells in the spleen.

In an embodiment of the present disclosure, Flt3L may be comprised at a concentration of 10 to 1,000 ng/ml in the medium, but is not limited thereto.

In another embodiment of the present disclosure, the culturing may be performed for 5 to 20 days, but is not limited thereto.

In still another embodiment of the present disclosure, the culturing may be performed for 10 days, but is not limited thereto.

In still another embodiment of the present disclosure, the treatment with interleukin-33 may be performed on the 3rd to 7th day from the starting day of culture, but is not limited thereto.

In still another embodiment of the present disclosure, the treatment with interleukin-33 may be performed at a concentration of 1 to 25 ng/ml, but is not limited thereto.

In still another embodiment of the present disclosure, wherein the treatment with interleukin-33 is performed at a concentration of 5 ng/ml on the 5th day from the starting day of culture, but is not limited thereto.

In still another embodiment of the present disclosure, the treatment with interleukin-33 may be performed at a point of time, in the stage of differentiation, at which the proportion of the number of CD103-positive dendritic cells relative to total dendritic cells is 30 to 100%, but is not limited thereto.

In still another embodiment of the present disclosure, the proportion of the number of CD103-positive type 1 myeloid-derived dendritic cells (cDC1) to total dendritic cells may be 70% or more, but is not limited thereto.

In accordance with another aspect of the present disclosure, there are provided CD103-positive dendritic cells prepared by the method according to the present disclosure.

In accordance with still another aspect of the present disclosure, there is provided a pharmaceutical composition for cancer immunotherapy, the composition comprising CD103-positive dendritic cells prepared by the method according to the present disclosure.

In an embodiment of the present disclosure, wherein the dendritic cells induce the expression of interferon gamma (IFN-γ), but is not limited thereto.

In another embodiment of the present disclosure, the dendritic cells may show an increase in the expression of Fc receptor, IgG, low affinity III (Fcgr3), but is not limited thereto.

In still another embodiment of the present disclosure, the dendritic cells may show an increase in the expression of at least one gene selected from the group consisting of cluster of differentiation 38 (CD38), integrin beta-3 (CD61), and T cell immunoreceptor with Ig and ITIM domains (Tigit), but is not limited thereto.

In accordance with still another aspect of the present disclosure, there is provided a kit for cancer immunotherapy, the kit including: (a) a first container comprising a composition comprising CD103-positive dendritic cells prepared by the method according to the present disclosure; (b) a second container comprising a tumor antigen; and (c) instructions stating that the composition in the first container and the antigen in the second container are mixed 12 to 48 hours before administration to a subject in need thereof.

In accordance with still another aspect of the present disclosure, there is provided a method for assessing immunogenicity of dendritic cells, the method includes measuring the proportion of CD103-positive dendritic cells.

In an embodiment of the present disclosure, the method may further include assessing the dendritic cells as having good immunogenicity if the proportion of the number of CD103-positive dendritic cells relative to total dendritic cells is 70% or more, but is not limited thereto.

In accordance with still another aspect of the present disclosure, there is provided a method for cancer immunotherapy, the method includes administering a pharmaceutical composition comprising CD103-positive dendritic cells to a subject in need thereof, wherein the CD103-positive dendritic cells are prepared by the method according to the present disclosure.

In accordance with still another aspect of the present disclosure, there is provided use of a composition comprising CD103-positive dendritic cells for cancer immunotherapy, wherein the CD103-positive dendritic cells are prepared by the method according to the present disclosure.

In accordance with still another aspect of the present disclosure, there is provided use of CD103-positive dendritic cells for the manufacture of a medicament for cancer immunotherapy, wherein the CD103-positive dendritic cells are prepared by the method according to the present disclosure.

### Advantageous Effects of Invention

According to the present disclosure, dendritic cell subsets newly differentiated by interleukin-33 (IL-33) were established, wherein the dendritic cells differentiated by IL-33 as above showed higher antigen-specific cytotoxic T cell inducing ability than dendritic cells of a control group, and strong anticancer immunity was induced by the dendritic cell subsets. Therefore, the present disclosure can enhance immunogenicity in a stage of differentiation, unlike existing methods of enhancing immunogenicity after completion of differentiation into dendritic cells. Accordingly, novel immune cell therapy is expected to be provided capable of dramatically increasing therapeutic effects through the preparation method of the present disclosure and the in vitro cultured dendritic cells prepared by the method.

### BRIEF DESCRIPTION OF DRAWINGS

FIG. 1 is a schematic diagram showing the time-sequential flow of preparation of in vitro cultured dendritic cells by using GM-CSF or Flt3L.

FIGS. 2A and 2B confirm subsets expressing CD103, showing a change in CD103+ cDC1 subsets for different times of IL-33 treatment in the preparation of FL-33-DC (FIG. 2A) and analysis results of CD103+ dendritic cell subsets in GM-DC, GM-33-DC, FL-DC, and FL-33-DC (FIG. 2B).

FIGS. 3A and 3B show analysis results of CD103+ dendritic cell subsets in FL-DC, FL-GM-DC, and FL-33-DC (FIG. 3A) and analysis results of antigen specific T cell division and activation inducing ability (FIG. 3B).

FIGS. 4A to 4D show the original data of transcriptome analysis of FL-DC, FL-GM-DC, and FL-33-DC (FIG. 4A), a graph comparing expression levels of genes that are high specifically for FL-33-DC (FIG. 4B), gene expression analysis confirmed by RT-PCR (FIG. 4C), and analysis results of expression levels of CD38, CD61, Tigit, and Fcgr3 proteins confirmed by flow cytometry (FIG. 4D).

FIGS. 5A to 5B show the results of analyzing tumor growth inhibitory effects of dendritic cell vaccines obtained by sensitizing GM-DC, GM-33-DC, FL-DC, FL-GM-DC, and FL-33-DC with OVA in mouse EG.7 tumor models (FIG. 5A) and the results of analyzing cytotoxic lymphocytes induced after vaccine administration (FIG. 5B).

FIGS. 6A to 6B show the results of analyzing tumor-derived nodule formation inhibitory effects of dendritic cell vaccines obtained by sensitizing FL-DC, FL-GM-DC, and FL-33-DC with OVA in mouse B16F10-OVA lung metastasis models (FIG. 6A) and the results of analyzing cytotoxic lymphocytes induced after vaccine administration (FIG. 6B).

FIGS. 7A to 7D show the proportion of CD103-positive dendritic cells in the spleen after the administration of GM-CSF or IL-33 to mice (FIG. 7A), a tumor growth graph after cytokine administration to EG.7 tumor models (FIG. 7B), the proportion of CD103-positive dendritic cells in the spleen of cytokine-administered tumor models (FIG. 7C), and the results of analyzing cytotoxic lymphocytes induced after cytokine administration (FIG. 7D).

FIG. 8 shows the results of analyzing, by flow cytometry, the protein expression levels of Fcgr3 and Fcgr4 in CD103-positive dendritic cells induced by IL-33.

### BEST MODE FOR CARRYING OUT THE INVENTION

The present inventors discovered that when dendritic progenitor cells are cultured in a medium comprising FMS-like tyrosine kinase 3 ligand (Flt3L) to induce the dendritic progenitor cells to differentiate into dendritic cells, the treatment with interleukin-33 (hereafter, “IL-33”) in a stage of differentiation into dendritic cells newly induced cluster of differentiation 103 (CD103)-positive dendritic cells with enhanced immunogenicity, leading to significant enhanced antitumor immunity, and therefor completed the present disclosure.

In an example of the present disclosure, in FL-33-DC (dendritic cells treated with IL-33 under an environment of Flt3L) unlike exciting FL-DC (dendritic cells cultured under an environment of Flt3L), CD103+ dendritic cell subsets were newly formed and the division of antigen-specific cytotoxic lymphocytes was induced more strongly (see Examples 2 to 4).

In another example of the present disclosure, when a vaccine comprising FL-33-DC was manufactured and administered to tumor models, the vaccine could inhibit tumor growth more effectively than existing GM-DC (dendritic cells cultured under an environment of GM-CSF) and GM-33-DC vaccine (dendritic cells treated with IL-33 under an environment of GM-CSF) as well as FL-DC vaccine (see Example 5).

In still another example of the present disclosure, FL-GM-DC (dendritic cells cultured under an environment of Flt3L and GM-CSF) known to generate CD103+ dendritic cells induced CD103+ dendritic cells, similar to FL-33-DC, but did not enhance antitumor immunity, unlike FL-33-DC. In contrast, the treatment with IL-33 performed in a stage of differentiation during the preparation of in vitro cultured dendritic cell vaccines induced immunogenic CD103+ dendritic cells and more effectively cytotoxic T cells, thereby showing a strong tumor inhibitory effect (see Example 5).

In still another example of the present disclosure, when a vaccine comprising FL-33-DC was prepared and administered to lung metastasis models, the vaccine significantly reduced the nodule formation and induced antitumor immunity most strongly compared with a FL-DC administration group (see Example 6).

In still another example of the present disclosure, IL-33 or GM-CSF was intraperitoneally administered to mice and then analyzed for the ability to induce CD103-positive dendritic cells in the spleen and the tumor inhibitory ability, and as a result, additional CD103-positive dendritic cells were induced by IL-33 or GM-CSF in the spleen of the tumor models in both the mouse groups, but antitumor immunity was strongly shown only by IL-33 (see Example 7).

In still another example of the present disclosure, the expressions of Fcgr3 and Fcgr4 were relatively high in CD103-positive dendritic cells induced by IL-33, unlike GM-CSF (see Example 8).

Accordingly, the present disclosure can provide a method for preparing CD103-positive dendritic cells, the method includes culturing dendritic progenitor cells in a medium comprising Flt3L to induce the dendritic progenitor cells to differentiate into dendritic cells, wherein a treatment with interleukin-33 (IL-33) is performed in a stage of differentiation into the dendritic cells.

As used herein, the term “dendritic cells (DCs)” refers to crucial specialized antigen-presenting cells (APCs) in the immune system, which can induce both the innate immune response and the adaptive immune response, wherein DCs can activate the naive cells without antigen contact and the memory immune response. DCs act as sentinels in their immature state to continuously patrolling for antigens. In general, APCs ingesting antigens present exogenous antigens mainly through major histocompatibility complex class II (MHC) (CD4+ T cell activation) and present endogenous antigens through MHC class I (CD8+ T cell activation). However, DCs have special ability to cross-present exogenous antigens through MHC class I as well as MHC class II. Therefore, DCs can activate CD4+ and CD8+ T cells more effectively.

DCs, which have undergone maturation after acquiring antigens, migrate to lymphoid organs and present antigens to naive T cells. T cell activation requires not only antigen presentation of APCs but also stimulation of costimulatory molecules (CD80, CD86, CD40, etc.) and inflammation promoting cytokines expressed on the APC surface. Fully mature DCs induce the differentiation of CD4+ T cells into T helper 1 (Th1) cells through these signals and also activate CD8+ T cells (cytolytic T lymphocytes). However, in the absence of APC stimulation on costimulatory molecules and inflammation promoting cytokines or in the presence of stimulation on immunosuppressive cytokines, CD4+ T cells differentiate into Th2 cells or regulatory T cells (Tregs).

Tumors and the tumor microenvironments (TMEs) directly induce DC dysfunction or hide tumor antigens and secrete large amounts of immunosuppressive cytokines to suppress cancer immune activity. To overcome these hurdles, research are being conducted for inducing anticancer T cell activity by again administering, into the body, a dendritic cell therapeutic agent in which autologous DCs loading antigens are trained in vitro so as to express costimulatory molecules and secrete inflammation promoting cytokines, so-called “dendritic cell cancer vaccine”, and the preparation method of the present disclosure has been completed by, during such research, newly discovering an in vitro culturing method for obtaining dendritic cells with enhanced immunogenicity and antitumor efficacy.

As used herein, the term “immunogenicity” refers to a property capable of inducing or maintaining an immune response, especially, inducing an immune response, when administered to a mammal, especially, when administered to a human individual.

The preparation method includes culturing dendritic progenitor cells in a medium comprising Flt3L to induce the dendritic progenitor cells to differentiate into dendritic cells. In one embodiment, culturing dendritic cells in the Flt3L-comprising medium was confirmed to produce dendritic cells with superior immunogenicity and antitumor efficacy compared with culturing using the generally widely used granulocyte-macrophage colony-stimulating factor (GM-CSF). Therefore, the medium for culturing dendritic cells according to the preparation method of the present disclosure may not comprise GM-CSF.

As used herein, the term “Flt3L”, an abbreviation of FMS-like tyrosine kinase 3 ligand, refers to a cytokine functioning as a growth factor that increases the number of immune cells by inducing the proliferation of hematopoietic progenitor cells to activate the hematopoietic progenitor cells, as well as the formation of new blood vessels. The Flt3L may be human-derived and may be a protein having the amino acid sequence of GenBank: AAA19825.1.

In the present disclosure, the Flt3L may be comprised at a concentration of 10 to 1,000 ng/ml in the medium, but is not limited thereto. In an embodiment, when dendritic cells are cultured for 10 days, a medium comprising Flt3L at a concentration of 100 ng/ml may be used.

In the present disclosure, the culturing may be conducted for 5 to 20 days, and preferably for 10 days, but is not limited thereto.

In the present disclosure, the treatment with interleukin-33 may be performed on the 3rd to 7th days from the starting day of culture, but is not limited thereto, and the treatment is preferably performed in a stage of differentiation. The term “stage of differentiation” is used to refer to all successive stages in which dendritic progenitor cells, for example, hematopoietic bone marrow progenitor cells, differentiate into immature dendritic cells or mature dendritic cells.

The “immature dendritic cells”, which are found in the early stage of differentiation, refers to dendritic cells that, like mature dendritic cells, do not express cell surface markers such as CD14, express HLA-DR, CD86, CD80, CD83 or CD40 at low levels, and express CD1a and CCR1, CCR2, CCR5 and CXCR1 at normal levels. The differentiation of immature dendritic cells is initiated by receiving various signals, and such differentiation leads to complete differentiation or partial differentiation depending on a combination of the received signals. The immature dendritic cells cannot activate T cells even by contact with T cells, due to low levels of inflammatory cytokines that are expressed.

The “mature dendritic cells” refer to cells formed by maturation of immature dendritic cells, and means cells in which cell surface markers, involved in B cell and T cell activation, for example, MHC class I or MHC class II (HLA-DR), cell adhesion factors (CD54, CD18, CD11), costimulatory molecules (e.g., CD86, CD80, CD83 or CD40) are expresses at higher levels or relatively increased levels compared with immature dendritic cells. Typically, mature dendritic cells express CCR7 and CXCR4 at high levels. In the mature dendritic cells, pro-inflammatory cytokines are released, and through mixed lymphocyte reactions, the proliferation of primitive allogeneic T cells and syngeneic T cells are increased and/or the expression and secretion of other immune response-related cytokines are increased.

In and embodiment, the treatment with interleukin-33 at a concentration of 5 ng/ml on the 5th day from the starting day of culture showed best effects in the formation of CD103+ dendritic cell subsets and the induction of antigen-specific cytotoxic T cell division. However, the preparation method of the present disclosure is not limited to the above-specified time and concentration, and the time and concentration for treatment with interleukin-33 may be appropriately adjusted by those skilled in the art according to the condition, cell number, and culture environments of dendritic cell progenitor cells. The treatment with interleukin-33 may be performed at an appropriate concentration, and for example, at a concentration of 1 to 25 nm, but is not limited thereto.

In the present disclosure, regarding the treatment time and concentration of interleukin-33, the treatment may be performed at a point of time at which the proportion of the number of CD103-positive dendritic cells relative to total dendritic cells can be 30 to 100%. For example, in the treatment with 5 ng/ml interleukin-33 under fixed culture conditions on the 2nd, 3rd, 4th, 5th, 6th, 7th, or 8th day from the starting day of culture, if the proportion of the number of CD103 dendritic cells relative to total dendritic cells is 30% or more by the treatment on only the 3rd to 7th days, the treatment with interleukin-33 may be performed by selecting any one day from the above period, and preferably, the treatment with interleukin-33 may be performed by selecting a point of time at which the proportion of CD103-positive dendritic cells is highest.

In the present disclosure, in the preparation method, the proportion of the number of CD103-positive type 1 myeloid-derived dendritic cells (cDC1) relative to total dendritic cells may be 70% or higher, but is not limited thereto.

In accordance with another aspect of the present disclosure, there may be provided CD103-positive dendritic cells prepared by the method according to the present disclosure.

In accordance with still another aspect of the present disclosure, there may be provided a pharmaceutical composition for cancer immunotherapy, the composition comprising CD103-positive dendritic cells prepared by the method according to the present disclosure.

In the present disclosure, the dendritic cells may induce the expression of interferon gamma (IFN-γ), but is not limited thereto.

In the present disclosure, the dendritic cells may have an increase in the expression of Fc receptor, IgG, low affinity III (Fcgr3), but is not limited thereto.

In the present disclosure, the dendritic cells may have an increase in the expression of at least one gene selected from the group consisting of cluster of differentiation 38 (CD38), integrin beta-3 (CD61), and T cell immunoreceptor with Ig and ITIM domains (Tigit), but are not limited thereto.

In the present disclosure, Fcgr3 plays an important role in host defense, such as phagocytosis of pathogens and regulation of immune cell differentiation. CD38 is a glycoprotein present on the surface of immune cells, such as natural killer cells, and CD61 is a cluster of differentiation expressed in platelets, leukocytes, and the like. Tigit is an immune receptor present in T cells or natural killer cells. An increase in the gene expression thereof means an excellent immune effect.

In accordance with still another aspect of the present disclosure, there is provided a method for cancer immunotherapy, the method includes administering a pharmaceutical composition comprising CD103-positive dendritic cells to a subject in need thereof, wherein the CD103-positive dendritic cells are prepared by the method according to the present disclosure.

In accordance with still another aspect of the present disclosure, there is provided use of a composition comprising CD103-positive dendritic cells for cancer immunotherapy, wherein the CD103-positive dendritic cells are prepared by the method according to the present disclosure.

In accordance with still another aspect of the present disclosure, there is provided use of CD103-positive dendritic cells for the manufacture of a medicament for cancer immunotherapy, wherein the CD103-positive dendritic cells are prepared by the method according to the present disclosure.

In accordance with still another aspect of the present disclosure, there may be provided a method for cancer immunotherapy, the method includes administering the interleukin-33 to a subject, but is not limited thereto. The interleukin-33 may form new CD103-positive dendritic cells in vivo, but is not limited thereto.

In the present disclosure, the in vivo injection of interleukin-33 may be performed at an appropriate dose, and the interleukin-33 may be administered at 5 to 500 μg/kg, but is not limited thereto. In addition, the in vivo administration of interleukin-33 may be performed daily for 4 to 11 days, but is not limited thereto.

In the present disclosure, the interleukin-33 can increase the expression of the Fc receptor, IgG, low affinity III (Fcgr3) or Fc receptor, IgG, low affinity IV (Fcgr4) gene in the CD103-positive dendritic cells that are newly formed in vivo, but is not limited thereto.

In the present disclosure, Fc receptor, IgG, low affinity III (Fcgr3) may be commonly increased in CD103-positive dendritic cells prepared in vitro after interleukin-33 treatment and CD103-positive dendritic cells derived from in vivo dendritic cells, but is not limited thereto.

As used herein, the term “pharmaceutical composition for cancer immunotherapy” may be used exchangeably with “cell therapeutic agent”, and refers to a medicine used for the purpose of treatment, diagnosis, and prevention through a series of processes, including changing biological characteristics of cells by in vitro multiplying and sorting living autologous, allogenic, and xenogeneic cells or by other methods to restore the functions of cells and tissues. Cell therapeutic agents have been managed as medicines in the US since 1993 and in Korea since 2002. Such cell therapeutic agents may be largely classified into two types: “stem cell therapeutic agents” for tissue regeneration and organic function recovery; “immune cell therapeutic agents” for immune response regulation, such as the inhibition of in vivo immune response or acceleration of immune response.

The pharmaceutical composition of the present disclosure may further comprise an appropriate carrier, excipient, and diluent, which are commonly used in the preparation of pharmaceutical compositions. The excipient may be, for example, at least one selected from the group consisting of a diluent, a binder, a disintegrant, a lubricant, an adsorbent, a humectant, a film-coating material, and a controlled-release additive.

The pharmaceutical composition according to the present disclosure may be formulated in the form of a powder, granules, a sustained-release granules, enteric granules, a liquid medicine, an ophthalmic solution, an elixir, an emulsion, a suspension, a spirit, a troche, aromatic water, lemonade, a tablet, a sustained-release tablet, an enteric tablet, a sublingual tablet, a hard capsule, a soft capsule, a sustained-release capsule, an enteric capsule, a pill, a tincture, a soft extract, a dry extract, a fluid extract, an injection, a capsule, a perfusate, a plaster, a lotion, a paste, a spray, an inhalant, a patch, a sterile injection, or a preparation for external use such as an aerosol by the methods commonly used, respectively, and the preparation for external use may have a formulation of such as a cream, a gel, a patch, a spray, an ointment, a plaster, a lotion, a liniment, a paste or a cataplasma, but the pharmaceutical composition may be preferably formulated into an injection.

Examples of the carrier, excipient, and diluent that may be comprised in the pharmaceutical composition according to the present disclosure may include lactose, dextrose, sucrose, oligosaccharides, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oils.

The injection according to the present disclosure may comprise a solvent, such as injectable sterile water, 0.9% sodium chloride injection, Ringer's solution, dextrose injection, dextrose+sodium chloride injection, PEG, lactated Ringer's solution, ethanol, propylene glycol, non-volatile oil-sesame oil, cottonseed oil, peanut oil, soybean oil, corn oil, ethyl oleate, isopropyl myristate, or benzene benzoate; a solubilizing aid, such as sodium benzoate, sodium salicylate, sodium acetate, urea, urethane, monoethylacetamide, butazolidine, propylene glycol, Tweens, nicotinamide, hexamine or dimethyl acetamide; a buffer, such as a weak acid and a salt thereof (acetic acid and sodium acetate), a weak base and a salt thereof (ammonia and ammonium acetate), an organic compound, a protein, albumin, peptones, or gums; an isotonic agent such as sodium chloride; a stabilizer, such as sodium bisulfite (NaHSO₃), carbon dioxide gas, sodium metabisulfite (Na₂S₂O₅), sodium sulfite (Na₂SO₃), nitrogen gas (N₂), or ethylenediaminetetraacetate; an antioxidant, such as 0.1% sodium bisulfide, sodium formaldehyde sulfoxylate, thiourea, disodium ethylenediaminetetraacetate, or acetone sodium bisulfite; a pain-relief agent, such as benzyl alcohol, chlorobutanol, procaine hydrochloride, glucose, or calcium gluconate; or a suspending agent, such as sodium CMC, sodium alginate, Tween 80, or aluminum monostearate.

The pharmaceutical composition according to the present invention is administered at a pharmaceutically effective amount. As used herein, the term “pharmaceutically effective amount” refers to an amount sufficient to treat a disease at a reasonable benefit/risk ratio applicable to medical treatment, and the effective dose level may be determined according to factors including the type of disease in a patient, the severity of disease, the activity of a drug, the sensitivity to a drug, the administration time, administration route, the excretion rate, the treatment period, and a drug used in combination, and other factors well known in the medical field.

The pharmaceutical composition according to the present disclosure may be administered as an individual therapeutic agent or administered in combination with other therapeutic agents, and may be administered sequentially or simultaneously with conventional therapeutic agents, and may be administered in either single or multiple doses. It is important to administer the composition at the minimum amount that can obtain the maximum effect without causing side effects, considering all the above-described factors, and this amount can be easily determined by a person skilled in the art.

The pharmaceutical composition of the present disclosure may be administered to a subject through various routes. All modes of administration may be contemplated, for example, administration may be performed by subcutaneous injection, intraperitoneal administration, intravenous injection, intramuscular injection, peri-spinal space (intrathecal) injection, sublingual administration, buccal administration, rectal insertion, vaginal insertion, ocular administration, ear administration, nasal administration, skin administration, or transdermal administration, and preferably, administration may be performed intradermally, intranodally, subcutaneously, intravenously, or directly intratumorally. However, optimal routes of administration have not yet been established.

The pharmaceutical composition of the present disclosure is determined according to the type of drug, as an active ingredient, along with various factors, such as a disease to be treated, a route of administration, patient's age, sex, and body weight, and the severity of a disease.

As used herein, the term “subject” refers to an object in need of treatment of a disease, and more specifically, refers to a mammal, such as a human or non-human primate, a mouse, a rat, a dog, a cat, a horse, or a cow.

As used herein, the term “administration” refers to providing a subject with a predetermined composition of the present disclosure by any appropriate method.

As used herein, the term “prevention” refers to any action that inhibits or delays the onset of a target disease, and the term “treatment” refers to any action that alleviates or beneficially changes a target disease and accompanied metabolic abnormalities by means of administration of the pharmaceutical composition according to the present disclosure.

In accordance with still another aspect of the present disclosure, there is provided a kit for cancer immunotherapy, the kit including: (a) a first container comprising a composition comprising CD103-positive dendritic cells prepared by the method according to the present disclosure; (b) a second container comprising a tumor antigen; and (c) instructions stating that the composition in the first container and the antigen in the second container are mixed 12 to 48 hours before administration to a subject in need thereof.

As used herein, the term “tumor antigen” refers to a marker that allows immune cells to recognize tumor as a foreign substance but not autologous cells, and mutation products of tumor (cells), products resulting from gene dysregulation in tumor (cells), and carcinogenic viral proteins, and the like may act as such antigens. Tumor antigens may be classified according to specificity into tumor-specific antigens (TSAs) specifically expressed only in tumor cells and tumor-associated antigens (TAAs). Tumor-specific antigens induce tumor-specific immune responses in the host, but tumor-associated antigens, which are expressed in normal cells as well as tumor cells, may not induce tumor-specific immune responses in the host, due to self-tolerance expression.

The mixing of the composition in the first container and the antigen in the second container is loading the antigen on dendritic cells. To load antigens on dendritic cells, a method of culturing peptides, proteins, and killed autologous/allogeneic cancer cells together is generally used. Short synthetic peptides (8-15 aa) are directly loaded onto MHC molecules of the surface of dendritic cells, but long synthetic peptides (28-35 aa), proteins, and cancer cells may be treated with peptides before being loaded onto MHC molecules. As for short synthetic peptides, the CD8+ T cell epitope for TAA is used in clinical trials, wherein the HLA haplotype of patients is required to be known and the peptides need to bind to a specific haplotype. Long synthetic peptides can be cross-presented through antigen processing in dendritic cells, thereby inducing CD4+ cell response as well as CD8+ T cell response and presenting antigens over a longer period of time.

In accordance with still another aspect of the present disclosure, there is provided a method for assessing immunogenicity of dendritic cells, the method includes measuring the proportion of CD103-positive dendritic cells.

In the present disclosure, the method may further include assessing the dendritic cells as having good immunogenicity if the proportion of the number of CD103-positive dendritic cells relative to total dendritic cells is 70% or more, but is not limited thereto. The CD103-positive dendritic cells are a marker for type 1 conventional DC (cDC1), and one example of the present disclosure confirmed that the higher the proportion of CD103+ dendritic cell subsets, the stronger the induction of antigen-specific T cells.

The terms and words used in this description and the appended claims are not to be interpreted in common or lexical meaning but, based on the principle that an inventor can adequately define the meanings of terms to best describe the invention, to be interpreted in the meaning and concept conforming to the technical concept of the present disclosure.

Hereinafter, preferable examples are set forth for better understanding of the present disclosure. However, the following examples are merely provided to facilitate understanding of the present disclosure, and the scope of the present disclosure is not limited by the following examples.

### EXAMPLES

### Example 1: Preparation of In Vitro Dendritic Cells

Bone marrow cells were isolated from mouse hind limbs and then cultured in an environment of 20 ng/ml GM-CSF. After replacement with fresh media on the 2nd day of culture, and dendritic cells cultured for 7 days based on the first day were named GM-DC. Unlike this, dendritic cells treated with 5 ng/ml IL-33 on Day 3 of culture of GM-DC were named GM-33-DC.

Meanwhile, dendritic cells prepared by culturing bone marrow cells in an environment of 100 ng/ml Flt3L for 10 days were named FL-DC. Additionally, dendritic cells cultured by adding 5 ng/ml IL-33 on Day 3-7 of culture of FL-DC were named FL-33-DC. GM-DC and GM-33-DC were subjected to cell analysis on Day 7, and FL-DC and FL-33-DC were subjected to cell analysis on Day 10 of culture.

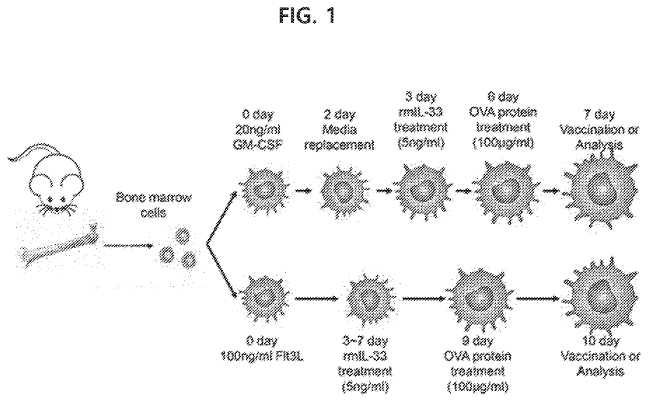

Dendritic cells for vaccine administration for investigating anticancer effects were treated with ovalbumin (hereinafter “OVA”) corresponding to the antigen one day before use, and administered to tumor model mice (see the schematic diagram of FIG. 1).

### Example 2: Increase in CD103-Expressing Subsets in FL-33-DC

In the preparation of FL-33-DCs according to Example 1 and FIG. 1, in order to find conditions in which CD103+ type 1 myeloid-derived dendritic cells (type 1 conventional DCs; hereinafter “cDC1”) are most induced, IL-33 treatment was performed at different days during the culture of FL-DC, and then the cells were harvested on Day 10 and analyzed.

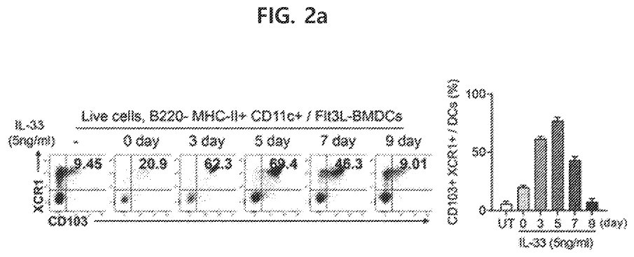

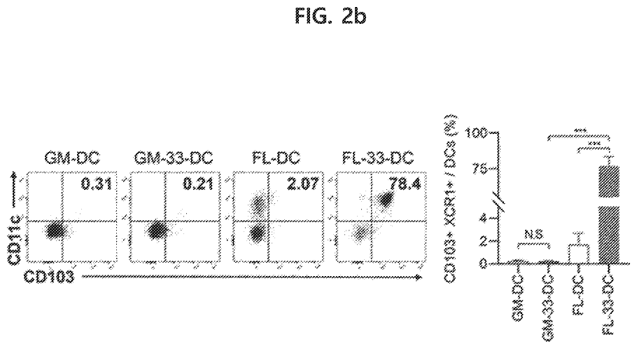

As a result of investigating XCR1 and CD103, cDC1 markers, as shown in FIGS. 2A and 2B, the proportion of CD103+ cDC1 was significantly increased for the IL-33 treatment on Day 3-7 of culture of FL-DC, and particularly, CD103+ cDC1 with high immunogenicity was most induced for the IL-33 treatment on Day 5 (FIG. 2A). Thus, the cells treated with IL-33 on Day 5 were used for FL-33-DC in the following example.

Meanwhile, GM-DC, GM-33-DC, FL-DC, and FL-33-DC prepared in Example 1 were analyzed for the proportion of CD103+ cDC1 with strong antitumor immunogenicity. CD103+ cDC1 was hardly induced in GM-DC, and the CD103+ cDC1 subsets were also not significantly induced in GM-33-DC with IL-33 addition. In FL-DC, XCR1 was expressed but CD103 was hardly expressed. In contrast, CD103+ cDC1 accounted for 75% or more of total dendritic cells in FL-33-DC (FIG. 2B).

The above results verified that when the IL-33 treatment was performed on Day 3-7 of culture during culture in an environment of Flt3L, a significantly higher proportion of CD103+ dendritic cell subsets was newly formed.

### Example 3: Enhanced Antigen-Specific T Cell Inducing Effect of FL-33-DC

It was confirmed in Example 2 that CD103+ dendritic cells were newly induced in FL-33-DC. Therefore, the induction of CD103+ cDC1 when GM-CSF was added during the preparation of FL-DC by the existing method was compared with that in FL-33-DC.

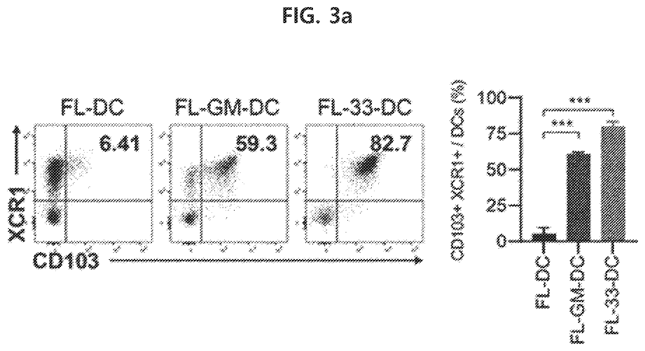

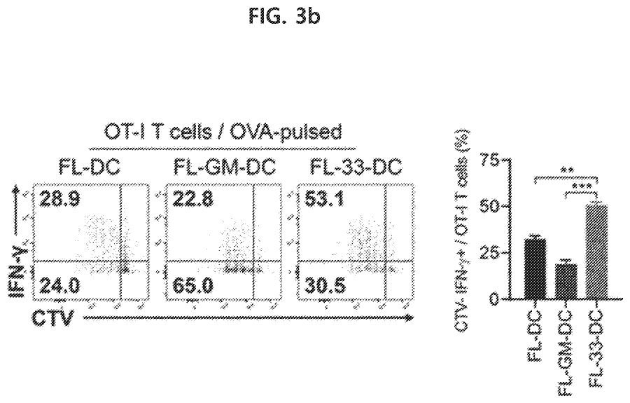

As a result, as shown in FIGS. 3A and 3B, the proportion of CD103+ cDC1 induced when CM-CSF treatment was performed on Day 5 of culture of FL-DC (hereinafter, named FL-GM-DC) was about 60%, which was lower than that in FL-33-DC (FIG. 3A).

To analyze whether the above change affected the antigen presenting ability of dendritic cells, FL-DC, FL-GM-DC, and FL-33-DC were treated with OVA protein and co-cultured with OT-1 mouse T cells having an OVA antigen-specific T cell receptor, and then these cells were examined for the proliferation of OT-1 T cells and the expression of IFN-γ, one of activating cytokines.

As a result, the proliferation ability of OT-1 T cells was higher in FL-33-DC and FL-GM-DC than FL-DC. However, unlike FL-33-DC effectively inducing IFN-γ, FL-GM-DC did not induce IFN-γ (FIG. 3B).

The above results confirm that CD103+ cDC1 was induced in both FL-33-DC and FL-GM-DC, but only FL-33-DC showed immunogenicity for T cells.

### Example 4: Expression of Immunogenicity-Related Specific Markers in FL-33-DC

To identify immunogenicity-related markers specifically expressed in FL-33-DC, FL-DC, FL-GM-DC, and FL-33-DC were subjected to transcriptome analysis.

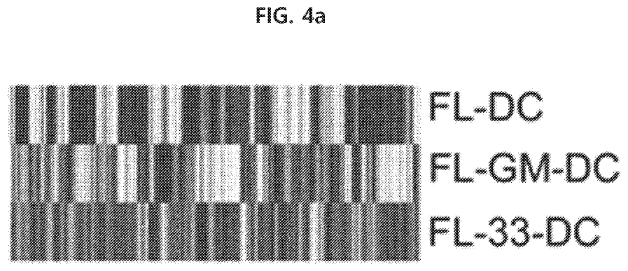

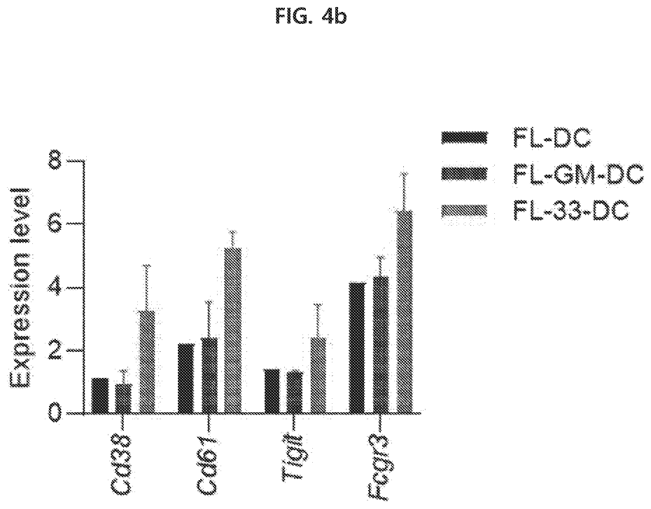

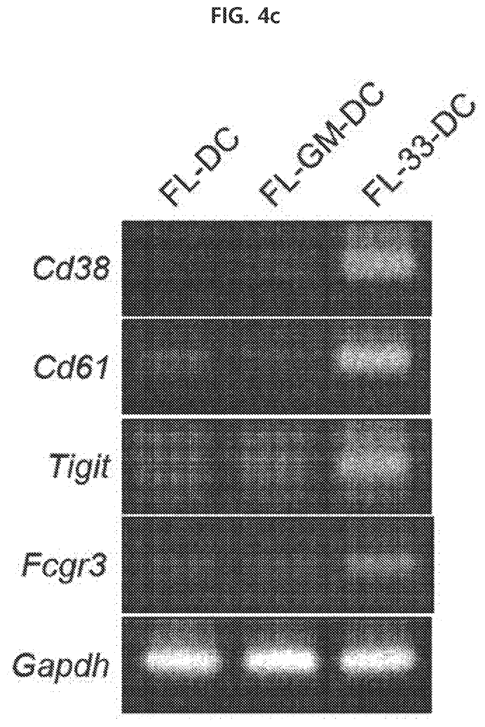

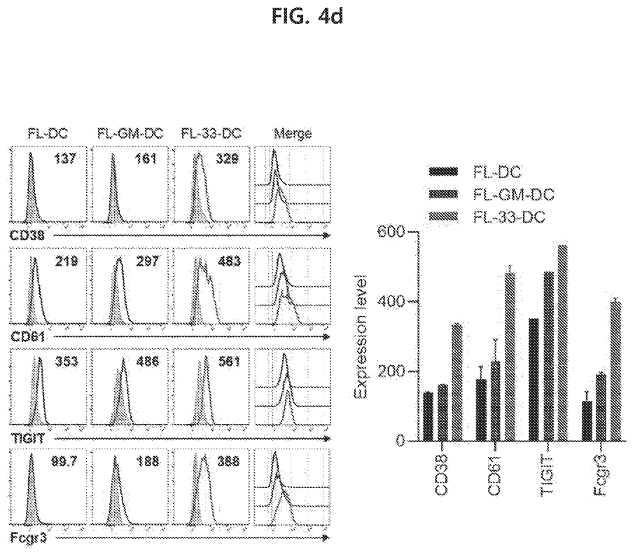

First, each type of dendritic cells (DCs) showed a specific gene expression pattern (FIG. 4A). Out of these, the expression levels of CD38, CD61, Tigit, and Fcgr3 were specifically high in FL-33-DC (FIG. 4B). Actually, the results of RT-PCR after RNA isolation confirmed that the above genes were especially highly expressed in FL-33-DC (FIG. 4C).

The four genes were analyzed at the protein level through flow cytometry.

As a result, as shown in FIG. 4D, CD38, CD61, and Fcgr3 were relatively highly expressed in FL-33-DC. The above results indicate that unlike FL-DC and FL-GM-DC, FL-33-DC specifically expresses immunogenicity-related molecules including such molecules and thus can enhance the ability to induce and activate T cells.

### Example 5: Notable Tumor Growth Inhibitory Ability of FL-33-DC

It was confirmed in Example 3 that FL-33-DC had a significantly higher ability to induce and activate antigen-specific T cells than FL-DC and FL-GM-DC, and it was confirmed in Example 4 that FL-33-DC relatively highly expressed immunogenicity-related CD38, CD61, and Fcgr3. Therefore, to investigate whether FL-33-DC could also be applied to tumor treatment, the dendritic cell vaccines prepared by the method shown in the schematic diagram of FIG. 1 were subcutaneously injected into EG.7 tumor-inoculated mice on the 3rd and 10th days after tumor inoculation, respectively, and tumor growth was monitored.

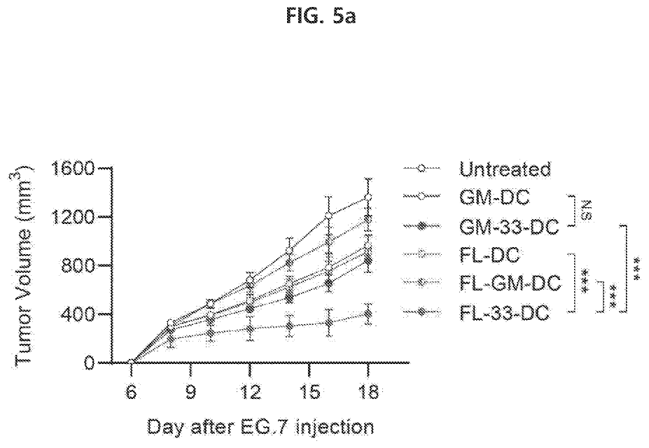

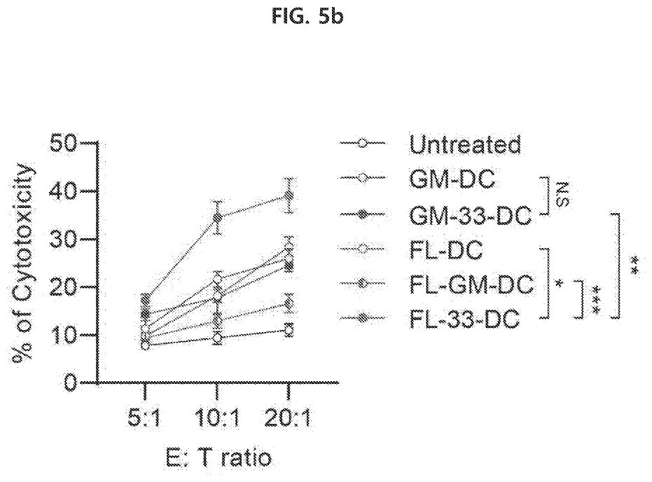

As a result, tumor growth was effectively inhibited significantly in the group administered with the FL-33-DC vaccine compared with the control group not administered with dendritic cells and the group administered with the FL-DC vaccine (FIG. 5A). FL-GM-DC showing the induction of CD103+ cDC1 similar to FL-33-DC did not inhibit tumor growth effectively (FIG. 5A). GM-DC used for existing immunotherapy showed a similar effect to FL-DC, and GM-33-DC with IL-33 addition did not show an additional therapeutic effect, unlike FL-33-DC (FIG. 5A).

In addition, as a result of analyzing antitumor immunity in the mice administered with these dendritic cell (DC) vaccines, antitumor immunity was most strongly induced in the mice administered with the FL-33-DC vaccine (FIG. 5B; CTL, cytotoxic T lymphocytes).

The above results confirmed that the enhanced tumor inhibitory effect of the FL-33-DC vaccine resulted from the effective induction of antigen-specific cytotoxic lymphocytes (CTL).

### Example 6: Ability of FL-33-DC to Inhibit Lung Metastatic Tumor Growth

Example 5 showed that the antitumor immunity was most strongly induced in the mice administered with the FL-33-DC vaccine, and thus it was investigated whether the vaccine could also be applied to lung metastasis models. The dendritic cell vaccines prepared by the method shown in the schematic diagram of FIG. 1 were subcutaneously injected into B16F10-OVA lung metastasis models on the 3rd and 10th days after tumor inoculation, respectively, and nodule formation in the lung was analyzed on the 16th day.

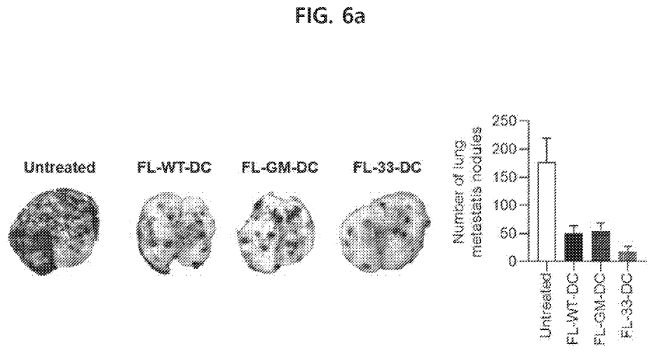

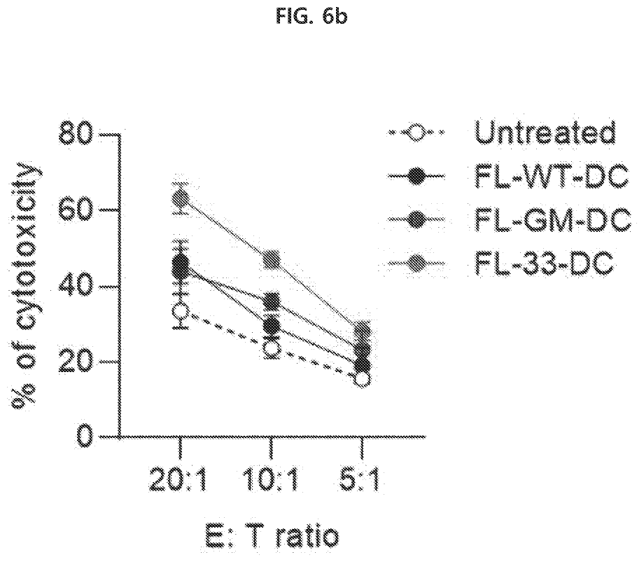

As a result, basically, the number of nodules was significantly decreased in the group administered with dendritic cells compared with the control group not administered with dendritic cells (FIG. 6A). The nodule formation was significantly reduced in the group administered with FL-33-DC compared with FL-DC (FIG. 6A). FL-GM-DC showing the induction of CD103+ cDC1 similar to FL-33-DC did not inhibit nodule formation compared with FL-33-DC (FIG. 6A).

In addition, as a result of analyzing antitumor immunity in the mice administered with these dendritic cell (DC) vaccines, antitumor immunity was most strongly induced in the mice administered with the FL-33-DC vaccine, similar to the results in FIG. 5B (FIG. 6B).

### Example 7: Tumor Inhibition and Induction of In Vivo CD103-Positive Dendritic Cells Through IL-33 Administration

IL-33 or GM-CSF was intraperitoneally administered to mice and then analyzed for the ability to induce CD103-positive dendritic cells in the spleen and the tumor inhibitory ability.

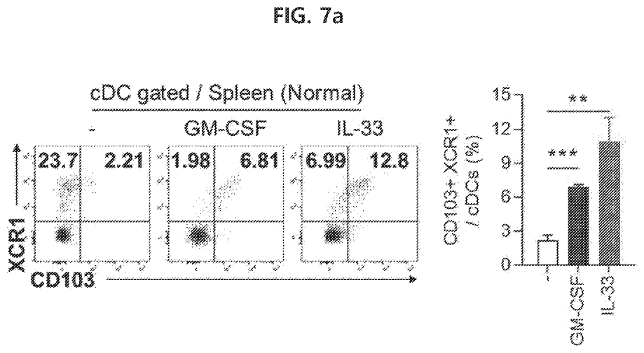

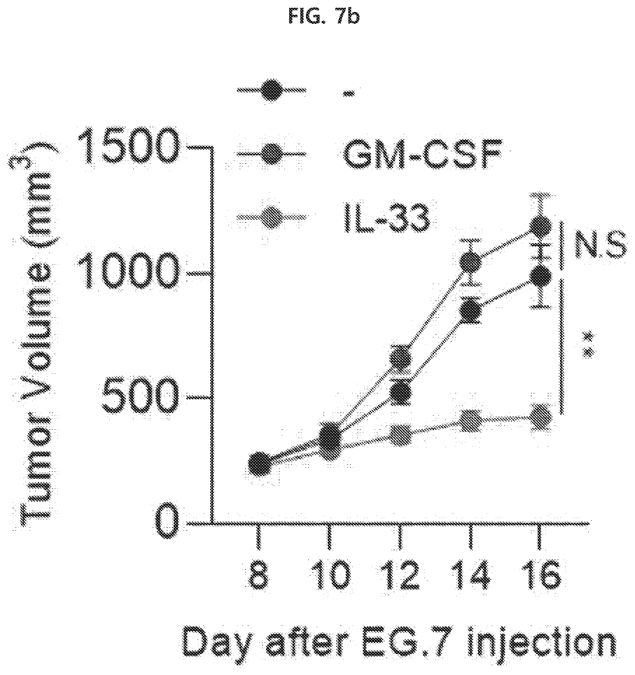

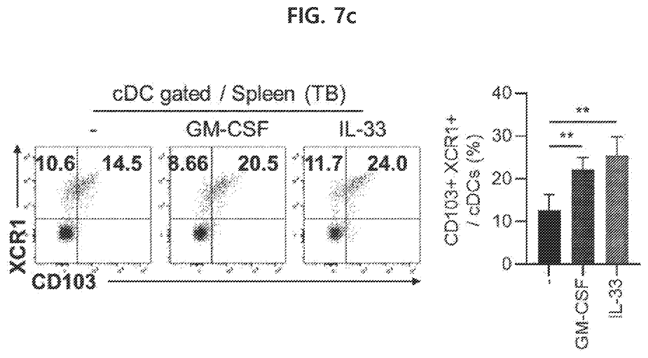

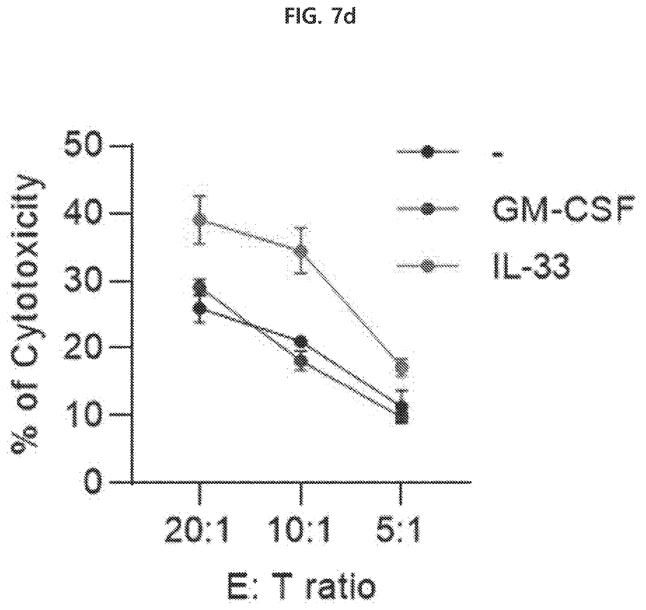

As a result, when IL-33 or GM-CSF was administered to tumor-free mice, CD103-positive dendritic cells were newly formed in the spleen (FIG. 7A). However, when anti-tumor immunity was induced in the tumor models, tumor was inhibited only by IL-33 (FIG. 7B). Additional CD103-positive dendritic cells were induced by IL-33 or GM-CSF in the spleen of the tumor models in both the mouse groups (FIG. 7C), but antitumor immunity was strongly shown only by IL-33 (FIG. 7D).

### Example 8: Markers for In Vivo IL-33-Induced CD103-Positive Dendritic Cells

In Example 7, both IL-33 and GM-CSF could induce CD103-positive dendritic cells, but showed completely different anti-tumor immune responses. Therefore, an attempt was made to identify markers capable of making a difference between the two.

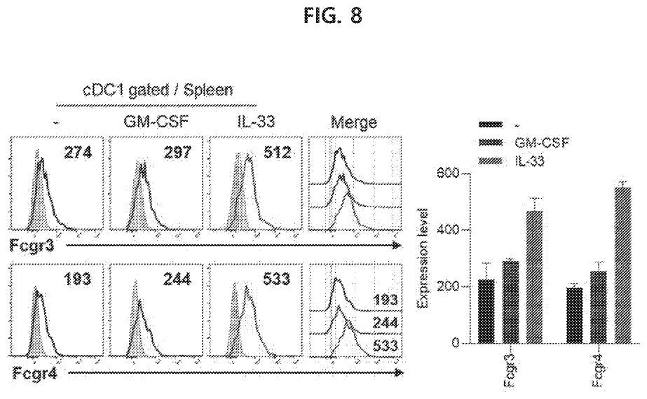

As a result, the expressions of Fcgr3 and Fcgr4 were relatively high in CD103-positive dendritic cells induced by IL-33, unlike GM-CSF (FIG. 8).

It was experimentally established that unlike FL-DC or FL-GM-DC, the inventive FL-33-DC highly expressed CD38, CD61, and Fcgr3 and increased CD103+ cDC1 with high immunogenicity, and CD103+ cDC1 expressing these molecules effectively induced antigen-specific cytotoxic T lymphocytes, and thus showed improved cancer treatment effect. Hence, FL-33-DC can be widely used as a cell therapeutic agent for anticancer immunotherapy.

Furthermore, if there is a method capable of directly administering IL-33 or increasing Fcgr3 and Fcgr4 in in vivo cDC1, such a method can also be used for immunotherapy. Particularly, Fcgr3 is commonly expressed in CD103+ cDC1 in the dendritic cells prepared in vitro by IL-33 and in the in vivo dendritic cells, and thus this molecule can be used for tumor treatment if the expression of this molecule can be enhanced.

The above description of the present disclosure is provided for the purpose of illustration, and it would be understood by those skilled in the art that various changes and modifications may be made without changing the technical idea and essential features of the present disclosure. Therefore, the embodiments and examples described above should be understood as being illustrative but not limitative in all aspects.

### INDUSTRIAL APPLICABILITY

According to the present disclosure, dendritic cell subsets newly differentiated by interleukin-33 (IL-33) were established, wherein the dendritic cells differentiated by IL-33 as above showed higher antigen-specific cytotoxic T cell inducing ability than dendritic cells of a control group, and strong anticancer immunity was induced by the dendritic cell subsets. Therefore, the present disclosure can enhance immunogenicity in a stage of differentiation, unlike existing methods of enhancing immunogenicity after completion of differentiation into dendritic cells. Accordingly, novel immune cell therapy is expected to be provided capable of dramatically increasing therapeutic effects through the preparation method of the present disclosure and the in vitro cultured dendritic cells prepared by the method, which are industrially applicable.

1. A method for preparing cluster of differentiation 103 (CD103)-positive dendritic cells, the method comprising culturing dendritic progenitor cells in a medium comprising FMS-like tyrosine kinase 3 ligand (FIt3L) to induce the dendritic progenitor cells to differentiate into dendritic cells, wherein a treatment with interleukin-33 (IL-33) is performed in a stage of differentiation into the dendritic cells.

2. The method of claim 1, wherein FIt3L is comprised at a concentration of 10 to 1,000 ng/ml in the medium.

3. The method of claim 1, wherein the culturing is performed for 5 to 20 days.

4. The method of claim 3, wherein the culturing is performed for 10 days.

5. The method of claim 1, wherein the treatment with interleukin-33 is performed on the 3rd to 7th day from the starting day of the culturing.

6. The method of claim 1, wherein the treatment with interleukin-33 is performed at a concentration of 1 to 25 ng/ml.

7. The method of claim 1, wherein the treatment with interleukin-33 is performed at a concentration of 5 ng/ml on the 5th day from the starting day of the culturing.

8. The method of claim 1, wherein the treatment with interleukin-33 is performed at a point of time, in the stage of differentiation, at which the proportion of the number of CD103-positive dendritic cells relative to total dendritic cells is 30 to 100%.

9. The method of claim 1, wherein the proportion of the number of CD103-positive type 1 myeloid-derived dendritic cells (cDC1) to total dendritic cells is 70% or more.

10. The method of claim 1, wherein the dendritic cells induce the expression of interferon gamma (IFN-γ).

11. The method of claim 1, wherein the dendritic cells show an increase in the expression of Fc receptor, IgG, low affinity III (Fcgr3).

12. The method of claim 1, wherein the dendritic cells show an increase in the expression of at least one gene selected from the group consisting of cluster of differentiation 38 (CD38), integrin beta-3 (CD61), and T cell immunoreceptor with Ig and ITIM domains (Tigit).

13. CD103-positive dendritic cells prepared by the method of claim 1.

14. A pharmaceutical composition for cancer immunotherapy, the composition comprising CD103-positive dendritic cells prepared by the method of claim 1.

15. (canceled)

16. The composition of claim 14, wherein the dendritic cells show an increase in the expression of Fc receptor, IgG, low affinity III (Fcgr3).

17. The composition of claim 14, wherein the dendritic cells show an increase in the expression of at least one gene selected from the group consisting of cluster of differentiation 38 (CD38), integrin beta-3 (CD61), and T cell immunoreceptor with Ig and ITIM domains (Tigit).

18. A kit for cancer immunotherapy, the kit comprising (a) to (c) below:
(a) a first container comprising a composition comprising CD103-positive dendritic cells prepared by the method of claim 1;
(b) a second container comprising a tumor antigen; and
(c) instructions stating that the composition in the first container and the antigen in the second container are mixed 12 to 48 hours before administration to a subject in need thereof.

19. A method for assessing immunogenicity of dendritic cells, the method comprising measuring the proportion of CD103-positive dendritic cells.

20. The method of claim 19, further comprising confirming if the proportion of the number of CD103-positive dendritic cells relative to total dendritic cells is 70% or more.

21. A method for cancer immunotherapy, the method comprising administering a pharmaceutical composition comprising CD103-positive dendritic cells to a subject in need thereof, wherein the CD103-positive dendritic cells are prepared by the method of claim 1.

22. (canceled)

23. (canceled)
